(19) United States
(12) Patent Application Publication
(10) Pub. No.: US 2002/0077258 A1
(43) Pub. Date: Jun. 20, 2002

(54) Multiphase soaps

(76) Inventors: Steffen Sonnenberg, Holzminden (DE); Marcus Ohrmann, Heinade (DE); Theodor Schmidt, Hoxter (DE); Rolf-Gunter Schmidt, Holzminden (DE)

Correspondence Address: BAYER CORPORATION, PATENT DEPARTMENT, 100 BAYER ROAD, PITTSBURGH, PA, 15205, US

(21) Appl. No.: 09/782,694

(22) Filed: Feb. 13, 2001

(30) Foreign Application Priority Data
Oct. 20, 2000 (DE) 10046469.6

(51) Int. Cl. A61K 7/50

(52) U.S. Cl. 510/141; 510/146; 510/152

(57) ABSTRACT

Multiphase soaps in which the individual phases are highly visible when viewed from above and from the side have high stability. Their use permits various scent experiences to be achieved during the washing operation.

### FIELD OF THE INVENTION

The invention relates to multiphase soaps in which the individual phases are highly visible when viewed from above and from the side, to their preparation and to their use for the application of different scent experiences during the washing operation.

### BACKGROUND OF THE INVENTION

DE-A 31 45 813 describes the preparation and use of picture and changing motif soaps. The preparation takes place by stamping various horizontal soap layers which have been prepared by means of an extruder. This process cannot be operated efficiently, meaning that use of these soaps is not possible in the mass consumer market. A particular disadvantage of this type of horizontally cut soap is the fact that the different horizontal soap layers cannot be distinguished or can be distinguished only with great difficulty by the consumer when viewed from a customary viewing angle of about 45° and above. This effect intensifies with increasing use time since the two phases become thinner as a result of being washed off.

This disadvantage also applies to the diverse horizontally cut soaps described in EP-A 0 366 209 and U.S. Pat. No. 5,198,140. U.S. Pat. No. 5,198,140 describes the preparation of an interlocked soap having increased strength. EP-A 0 366 209 describes the preparation of horizontal multiphase soaps by a casting process. However, casting processes are only suitable for the preparation of small numbers of bars, but not for the production of soaps for the mass consumer market.

EP-A 0 594 077 describes the preparation of spiral-shaped multiphase soaps which are prepared using a special compression head following radial rotation of the soap strand. Particularly in cases where different soap bases are used, the stability of the type of soap is limited in its application by the many phase boundaries.

DE-A 1 924 980 describes a process for the preparation of a multiphase soap with one or more sheaths which surround a core. This type of soap cannot be differentiated visually by the consumer from a normal single-phase soap before use and also in between during use, as a result of which there is no applications-related advantage.

The same also applies to soaps prepared in accordance with JP-A 62/48799. Here, a multilayer round strand is produced.

Soaps are also known in which a vertical cut in the transverse or in the longitudinal direction of the soap separates the two soap phases (e.g., JP 1-247499). In this type of soap, both phases are visible at the same time. However, the vertical type of soap, during use by the consumer and during continuous storage, exhibits the decisive disadvantage of lower stability of the overall bar of soap. Because of the small and straight contact areas, a vertically cut soap may break even as a result of the soap simply falling to the ground. In particular, when different soap formulations are used for the individual parts of the soap, shrinkage and drying out can lead to breaking of the soap. Also, when different soap formulations are used, the strength of the diagonally cut soap, in contrast to vertically joined soap, is ensured during use by the consumer over the entire use period. As a result, it may, in the future, also be possible to combine less expensive soap formulations with more expensive soap formulations, or to combine different soap formulations, which are incompatible with one another because of shrinkage, for the preparation.

EP-A 0 545 716 describes the preparation of a multi-dimensionally curved two-phase soap. By using the casting process, a two-phase soap is produced, which is not suitable for the mass consumer market due to the costly preparation. Since the soap here is a cast soap in which no pressure is subsequently exerted in the form of stamping, the durability of this type of soap is limited during daily use.

Marbled soaps are also known (DE-A 2 455 982, DE 2 431 048, U.S. Pat. No. 1,587,430 and DT 1 953 916). Here, differently colored soap phases are mixed intensively with one another using special pressing cylinders or screw presses, such that a marbled effect arises. Furthermore, the color can also be injected into the soap stream during the preparation of the soap. The soaps here consist of one phase.

A further multicolored single-phase soap is described in U.S. Pat. No. 4,435,310. Here, by injecting color during the preparation of the soap strands and by manually turning the extruder head, a multicolored sinusoidal soap is obtained from one cake.

### SUMMARY OF THE INVENTION

The objects of the present invention were multiphase soaps in which the different phases may have different ingredients which, during use, have a stability comparable with that of a single-phase soap. In particular, it is possible for the different phases to comprise different perfume oils so that, during use, different successive scent experiences are possible.

We have found multiphase soaps comprising two or more phases which are characterized in that the latter are highly visible when viewed from above and from the side.

The multiphase soaps according to the present invention exhibit superproportional strength, which virtually corresponds with the stability of a single-phase soap.

### BRIEF DESCRIPTION OF THE DRAWINGS

FIG. 1A shows a top view of a single-phase standard soap.

FIG. 1B shows a perspective view of the soap of FIG. 1A.

FIG. 2A shows a top view of a soap with horizontal soap layers according to DE-A 3 154 813

FIG. 2B shows a perspective view of the soap of FIG. 2A.

FIG. 3A shows a top cross sectional view of a two-phase soap according to the present invention.

FIG. 3B shows a perspective view of the soap of FIG. 3A.

FIG. 4A shows a top view of a two-phase soap with a longitudinal section.

FIG. 4B shows a perspective view of the two-phase soap of FIG. 4A.

FIG. 5A shows a top view of a multiphase soap with different cutting angles.

FIG. 5B shows a side view of the multiphase soap of FIG. 5A.

FIG. 6A shows a top view of a multiphase soap with different cutting angles of the longitudinal type.

FIG. 6B shows a side view of the multiphase soap of FIG. 6A.

FIG. 7A shows a top view of a longitudinal section through a two-phase soap.

FIG. 7B shows a perspective view of the soap of FIG. 7A.

FIG. 8A shows a top view of a diagonal section of a two-phase soap

FIG. 8B shows a perspective view of a diagonal section of the soap of FIG. 8A.

FIG. 9A shows a top view of a cross section of a two-phase soap.

FIG. 9B shows a perspective view of a cross section of the soap of FIG. 9A.

FIG. 10A shows a top view of a transverse type, displaced section through a two-phase soap.

FIG. 10B shows a perspective view of the soap of FIG. 10A.

FIG. 11 shows a measuring device for fracture tests.

FIG. 12A shows a top view of a three-phase soap with a displaced section.

FIG. 12B shows a perspective view of the soap of FIG. 12A.

### DETAILED DESCRIPTION OF THE INVENTION

Multiphase soaps are preferred in which each phase is visible in the vertical, longitudinal and transverse projection to at least 15%, based on the overall projected area.

Even more preferred are multiphase soaps in which each phase is visible in the vertical, longitudinal and transverse projection to at least 20%, based on the overall projected area.

In a preferred embodiment of the multiphase soaps according to the present invention, adjacent phase areas are cut diagonally and cambered towards one another. The cambering is achieved during the preparation using pressure. Multiphase soaps with cambered points of intersection have particular stability.

The multiphase soaps according to the present invention preferably comprise two phases which have a different composition.

The soap bases for the multiphase soaps according to the present invention are known per se (Soaps and Detergents, Luis Spitz, 0-935315-72-1 and Production of Soap, D. Osteroth, 3-921956-55-2). For example, soap bases such as alkali metal soaps consisting of animal and/or vegetable substances, syndets consisting of synthetic surfactants or combinations of the two may be used for the multiphase soaps according to the present invention.

The soap base can comprise, as further ingredients, for example, perfume oils, cosmetic ingredients, dyes and further additives.

It is preferred to add different perfume oils which are released successively and, during the washing operation, convey different, successive scent experiences to the user, or, as a result of the simultaneous release of the individual perfume oils, form a new more intensive scent.

Examples of fragrances in the perfume oils for the multiphase soaps according to the present invention are given, for example, in S. Arctander, Perfume and Flavor Materials, Vol. I and II, Montclair, N.J., 1969, published privately or K. Bauer, D. Garbe and H. Surburg, Common Fragrance and Flavor Materials, 3^{rd}. Ed., Wiley-VCH, Weinheim 1997.

Individual examples which may be mentioned are:

Extracts from natural raw materials such as essential oils, concretes, absolutes, resins, resinoids, balsams, tinctures, such as, for example, ambergris tincture; amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; corianda oil; costus root oil; cumin oil; cypress oil; Davana oil; dill herb oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camomile blue oil; camomile Roman oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lavage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtenol; clove leaf oil; clove flower oil; neroli oil; olibanum absolute; olibanum oil; opopanax oil; orange-flower absolute; orange oil; origanum oil; palmarosa oil; patchouli oil; perilla oil; peruvian balsam oil; parsley leaf oil; parsley seed oil; petitgrain oil; peppermint oil; pepper oil; pimenta oil; pine oil; pennyroyal oil; rose absolute; rosewood oil; rose oil; rosemary oil; dalmation sage oil; Spanish sage oil; sandalwood oil; celery seed oil; spike lavender oil; Japanese anise oil; styrax oil; tagetes oil; fir needle oil; tea-tree oil; turpentine oil; thyme oil; Tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom;

individual fragrances from the group of hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;

of aliphatic alcohols, such as, for example, hexanol; octanol; 3-octanol; 2,6-dimethylheptanol; 2-methylheptanol, 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol; of aliphatic aldehydes and 1,4-dioxacycloalken-2-ones thereof, such as, for example, hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyl oxyacetaldehyde;

of aliphatic ketones and oximes thereof, such as, for example, 2-heptanone; 2-octanone; 3-octanone; 2-nonanone; 5-methyl-3-heptanone; 5-methyl-3-heptanone oxime; 2,4,4,7-tetramethyl-6-octen-3-one; of aliphatic sulphur-containing compounds, such as, for example, 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

of aliphatic nitriles, such as, for example, 2-nonenenitrile; 2-tridecenenitrile; 2,12-tridecenenitrile; 3,7-dimethyl-2,6-octadienenitrile; 3,7-dimethyl-6-octenenitrile;

of aliphatic carboxylic acids and esters thereof, such as, for example, (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate, isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl 2-isoamyloxyacetate; methyl 3,7-dimethyl-2,6-octadienoate;

of acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;

of acyclic terpene aldehydes and ketones, such as, for example, geranial; neral; cirtonellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

of cyclic terpene alcohols, such as, for example, menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates, 3-methyl-2-butenoates thereof;

of cyclic terpene aldehydes and ketones, such as, for example, menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-iron; alpha-damascone; beta-damascone; beta-damascenone; delta-damascone; gammadamascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);

of cyclic alcohols, such as, for example, 4-tert-butylcyclohexanol; 3,3,5-trimethylcyclohexanol; 3-isocamphylcyclohexanol; 2,6,9-trimethyl-Z2,Z5,-E9-cyclododecatrien-1-ol; 2-isobutyl-4-methyltetrahydro-2H-pyran-4-ol;

of cycloaliphatic alcohols, such as, for example, alpha-3,3-trimethyl-cyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclo-hexyl)hexan-3-ol;

of cyclic and cycloaliphatic ethers, such as, for example, cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;

of cyclic ketones, such as, for example, 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclo-hexanone; 4-tert-pentylcyclohexanone; 5-cyclohexadecen-1-one; 6,7-dihydro-1,1,2,3,3-pentamethyl-4(5H)-indanone; 5-cyclohexadecen-1-one; 8-cyclohexadecen-1-one; 9-cycloheptadecen-1-one; cyclopentadecanone;

of cycloaliphatic aldehydes, such as, for example, 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

of cycloaliphatic ketones, such as, for example, 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl 2,4-dimethyl-3-cyclohexen-1-yl ketone;

of esters of cyclic alcohols such as, for example, 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;

of esters of cycloaliphatic carboxylic acids, such as, for example, allyl 3-cyclohexyl-propionate; allyl cyclohexyloxyacetate; methyl dihydrojasmonate; methyl jasmonate; methyl 2-hexyl-3-oxocyclopen-tanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolan-2-acetate;

of aromatic hydrocarbons, such as, for example, styrene and diphenylmethane;

of araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

of esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alphatrichloromethylbenzyl acetate; alpha, alpha-dimethylphenylethyl acetate; alpha,alpha-dimethylphenylethyl butyrate; cinnamyl acetate; 2-phenoxyethyl isobutyrate; 4-methoxybenzyl acetate; of araliphatic ethers, such as, for example, 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

of aromatic and araliphatic aldehydes, such as, for example, benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

of aromatic and araliphatic ketones, such as, for example, acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

of aromatic and araliphatic carboxylic acids and esters thereof, such as, for example, benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

of nitrogen-containing aromatic compounds, such as, for example, 2,4,6-trinitro-1,3-dimethyl-5-tert-butylbenzene; 3,5-dinitro-2,6-dimethyl-4-tert-butylacetophenone; cinnamonitrile; 5-phenyl-3-methyl-2-pentenenitrile; 5-phenyl-3-methylpentanenitrile; methyl anthranilate; methyl N-methylanthranilate; Schiff bases of methyl anthranilate with 7-hydroxy-3,7-dimethyl-octanal; 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine;

of phenols, phenyl ethers and phenyl esters, such as, for example, estragole; anethole; eugenole; eugenyl methyl ether; isoeugenole; isoeugenyl methyl ether; thymole; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

of heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy4H-pyran-4-one;

of lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The perfume oils are generally added to the soap base in an amount of from 0.05 to 5% by weight, preferably from 0.1 to 2.5% by weight, more preferably from 0.2 to 1.5% by weight, based on the soap base.

The perfume oils may be added in liquid form, neat or diluted with a solvent for perfuming the soap base. Suitable solvents for this purpose are, for example, ethanol, isopropanol, diethylene glycol monoethyl ether, glycerol, propylene glycol, 1,2-butylene glycol, dipropylene glycol, diethyl phthalate, triethyl citrate, isopropyl myristate, and etc.

In addition, the perfume oils for the multiphase soaps according to the present invention can be adsorbed on a carrier which serves both to distribute the fragrances finely within the product and to release them in a controlled manner during use. Such carriers can be porous inorganic materials, such as light sulphate, silica gels, zeolites, gypsums, clays, clay granules, gas concrete, etc. or organic materials such as woods and cellulose-based substances.

The perfume oils for the multiphase soaps according to the present invention can also be microencapsulated, spray dried, in the form of inclusion complexes or in the form of extrusion products and be added in this form to the soap base to be perfumed.

The properties of the perfume oils modified in this way can optionally be further optimized by so-called “coating” with suitable materials with regard to a more targeted fragrance release, for which purpose preference is given to using wax-like polymers such as, for example, polyvinyl alcohol.

The microencapsulation of the perfume oils can, for example, be carried out by the so-called coacervation method using capsule materials made from, for example, polyurethane-like substances or soft gelatins. The spray-dried perfume oils can, for example, be prepared by spray drying an emulsion or dispersion comprising the perfume oil, where the carriers used can be modified starches, proteins, dextrin and vegetable gums. Inclusion complexes can be prepared, for example, by introducing dispersions of the perfume oil and cyclodextrins or urea derivatives into a suitable solvent, e.g., water. Extrusion products can be obtained by melting the perfume oils with a suitable wax-like substance and by extrusion with subsequent solidification, optionally in a suitable solvent, e.g., isopropanol.

The perfume oils may be released simultaneously or successively during use. In particular, perfume oils are used which are released successively as a result of the targeted application of the individual soap phases.

Cosmetic ingredients for the multiphase soaps according to the present invention are known per se (Soaps and Detergents, Luis Spitz, 0-935315-72-1 and Production of Soap, D. Osteroth, 3-921956-55-2). For example, the cosmetic ingredients below may be mentioned: Cooling active ingredients, such as, e.g., menthol and menthol derivatives, warming active ingredients, such as, e.g., capsaicin, UV filters, such as, e.g., Neo Heliopans® to protect against discoloration of the soap or protect against solar irradiation on the skin, vitamins, such as, e.g., vitamins A and E for vitalizing the skin, vegetable waxes and oils, such as, e.g., cocoa butter, almond oil, avocado oil and jojoba oil, for improving the feel on the skin, plant extracts, moisturizers, minerals, antidandruff active ingredients, such as, e.g., Crinipan®, active ingredients, such as, e.g., deodorizing active ingredients sodium carbonate, triclosan and triclocarban.

The following additives are also known: dyes, such as, e.g., titanium dioxide, the addition of stabilizers such as, e.g., DTPA and EDTA, the addition of antioxidants such as BHT, the addition of filling materials, such as, e.g., starch and cellulose, the addition of hardeners, such as, e.g., sodium chloride and sodium sulphate.

We have also found a process for the preparation of the multiphase soaps according to the present invention, which is characterized in that the individual phases in the form of soap strands are diagonally cut precisely at an angle of from 14° to 70°, preferably 30 to 55°, and the points of intersection are joined under pressure so that cambering arises at the points of intersection.

The phases at the points of intersection are preferably joined using a pressure of from 4 to 10 bar or with a pressing weight of 1.0 to 2.0 t. A particularly preferred cambering of the points of intersection arises under these conditions.

The soap strands are prepared in a manner known per se: after the addition of additives to the soap base, milling and extrusion are carried out. Furthermore, during industrial production, the additives may be added during extrusion (Soaps and Detergents, Luis Spitz, 0-935315-72-1 and Production of Soap, D. Osteroth, 3-921956-55-2).

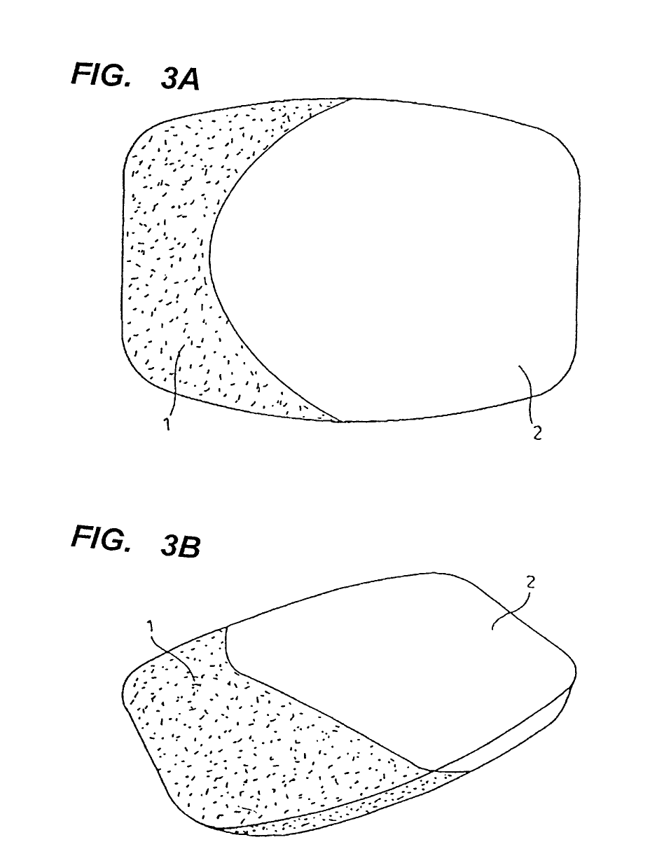

The multiphase soaps according to the present invention can be illustrated using the example of FIG. 3: FIG. 3 shows the multiphase soap according to the present invention in perspective view (FIG. 3B) and when viewed from above (FIG. 3A). The different phases are labelled 1 and 2. The diagram also depicts the cambering of the two phases.

The multiphase soaps according to the present invention surprisingly have high stability and can be prepared favorably in large bar numbers.

The invention is further illustrated but is not intended to be limited by the following examples in which all parts and percentages are by weight unless otherwise specified.

### EXAMPLES

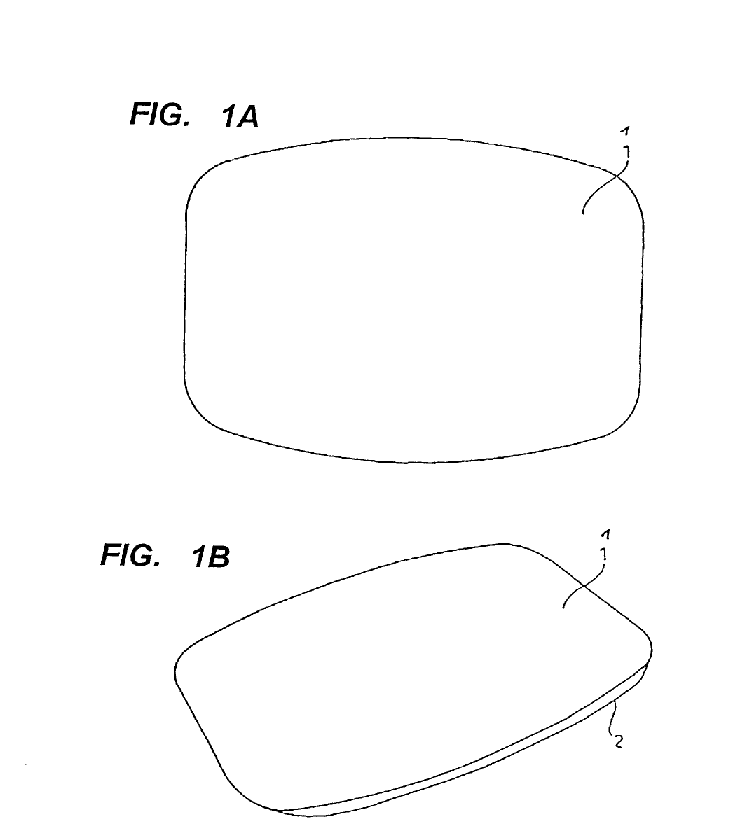

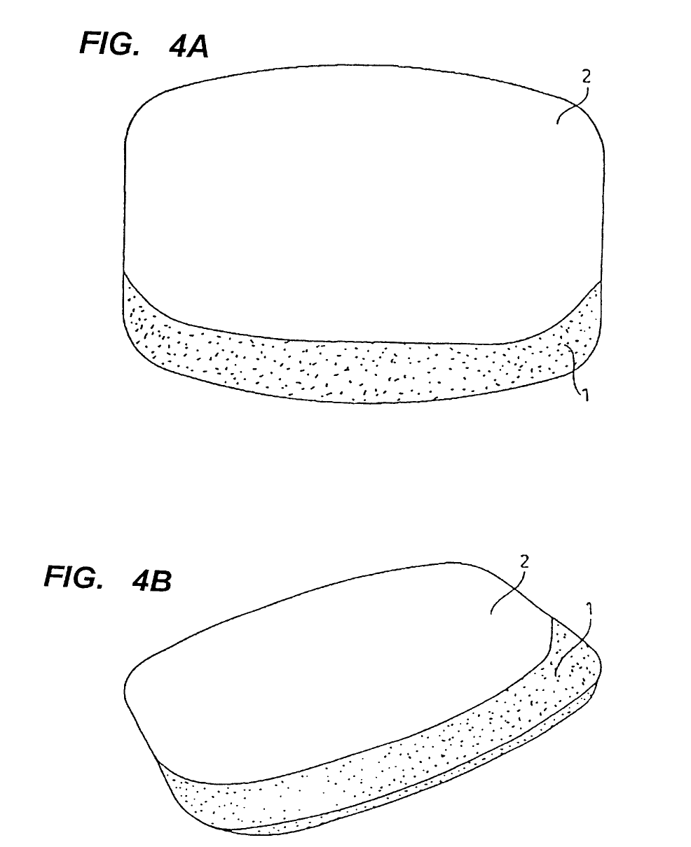

Using the example of a rounded standard soap shape (FIGS. 1A and 1B perspective view of a standard soap) with a length of 7.4 cm, a height of 1.8 cm and a width of 5.4 cm, the intersection shapes according to the present invention in the longitudinal and in the transverse direction of the multiphase soap are described below (FIGS. 3A and 3B transverse type central section, perspective view and FIGS. 4A and 4B longitudinal type central section, perspective view). This is only one application example since the different soap shapes can vary significantly in their length, height and width.

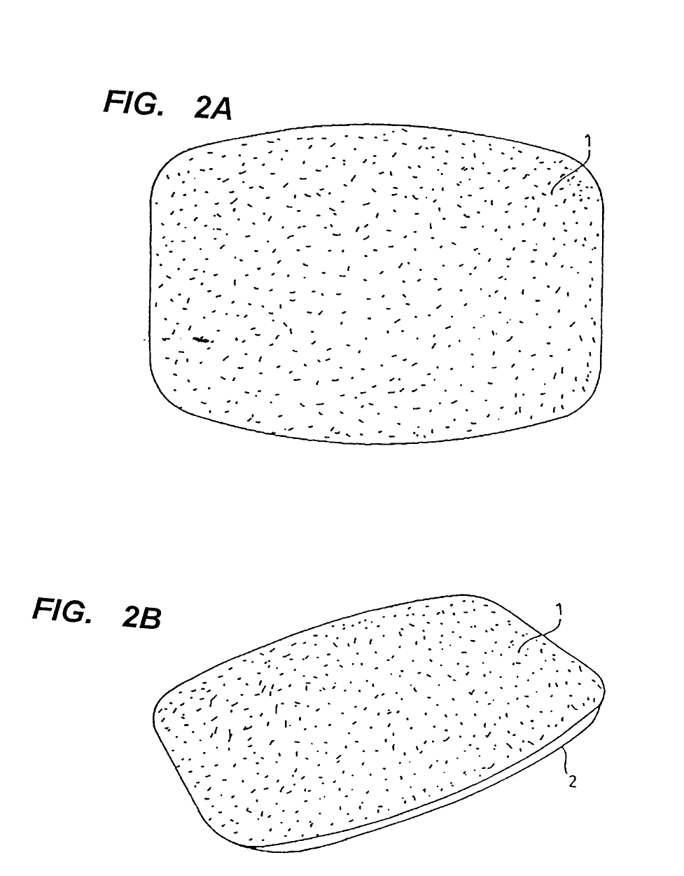

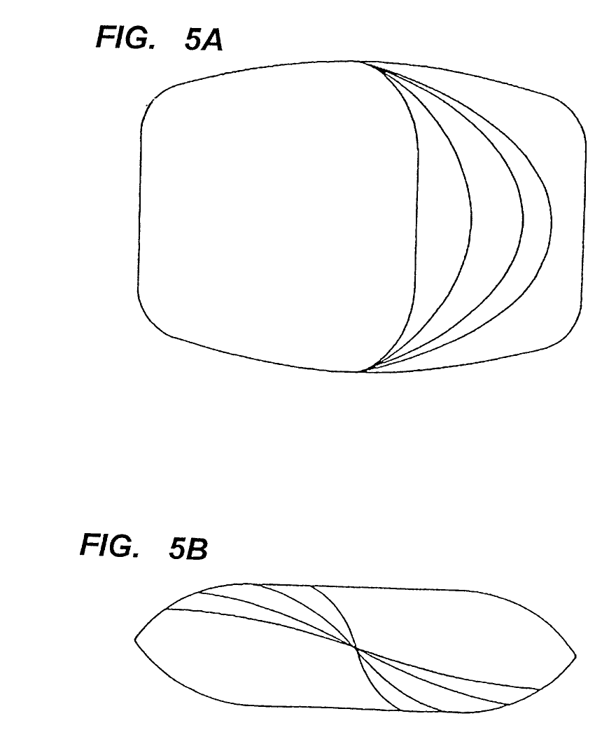

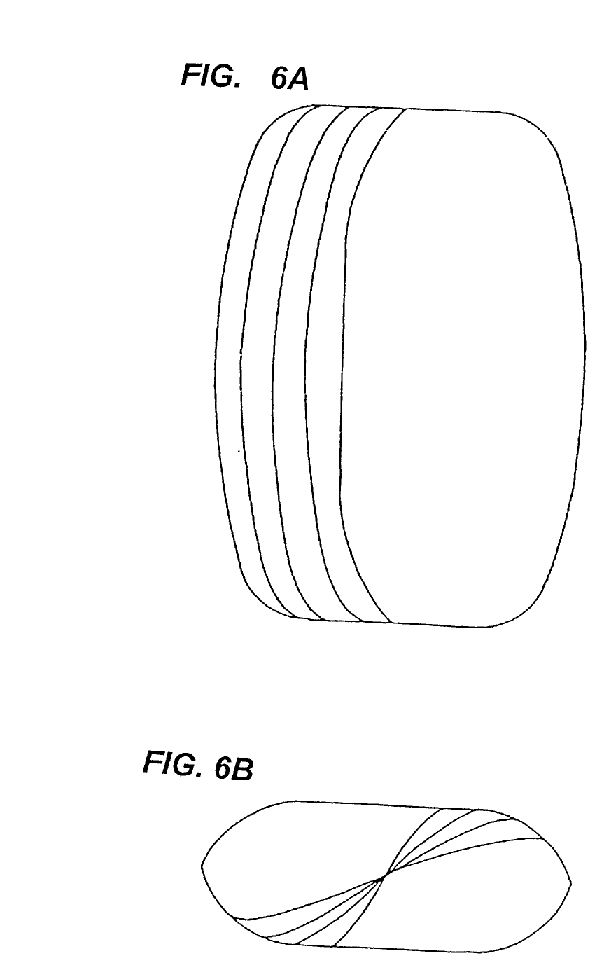

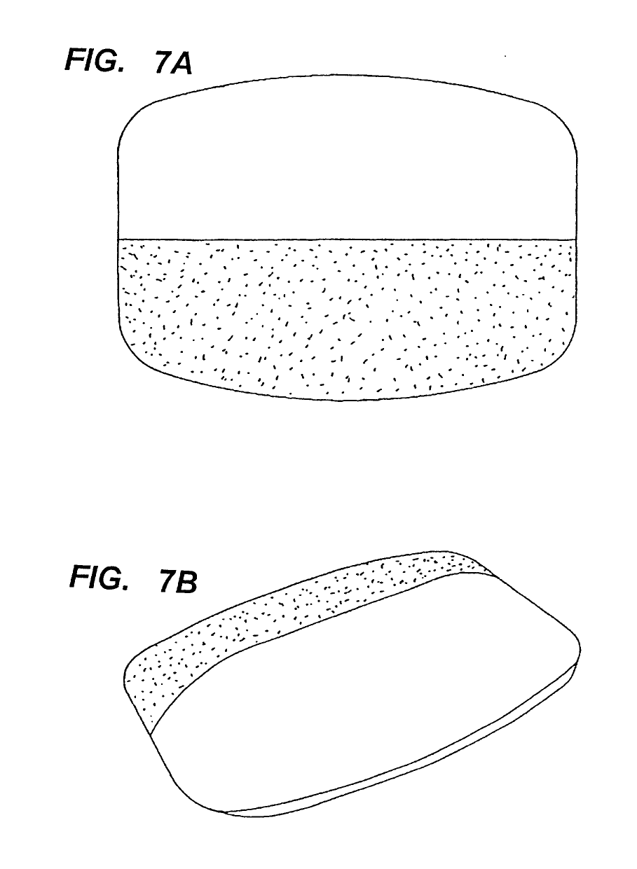

A soap bar of standard shape can be divided into two or more parts. For the purposes of the present invention, this division is in the direction of the longitudinal or transverse axis of the bar of soap at an angle between 0 and 90°. From this arise bars of soap with different types of intersection (FIGS. 5A and 5B, transverse type central section and FIGS. 6A and 6B, longitudinal type central section) and varying ratios of the viewing areas of the individual soap phases. The actual cutting angle arises as a result of the area ratios to be achieved which are necessary for distinguishing the individual soap phases. Depending on the ratio of the height to the length of the standard soap described of about 3.44, an angle of the cutting surface between the soap phases of about 14° to 60° then arises for the transverse type and an angle of about 20° to 70° for the longitudinal type. This new cutting angle varies depending on the shape of the soap and is industrially a new type of requirement during the preparation of the soap and differs significantly from the customary cutting angles of 0° (horizontal section, FIGS. 2A and 2B) or 90° (vertical section, FIGS. 7A-7B, 8A-8B and 9A-9B).

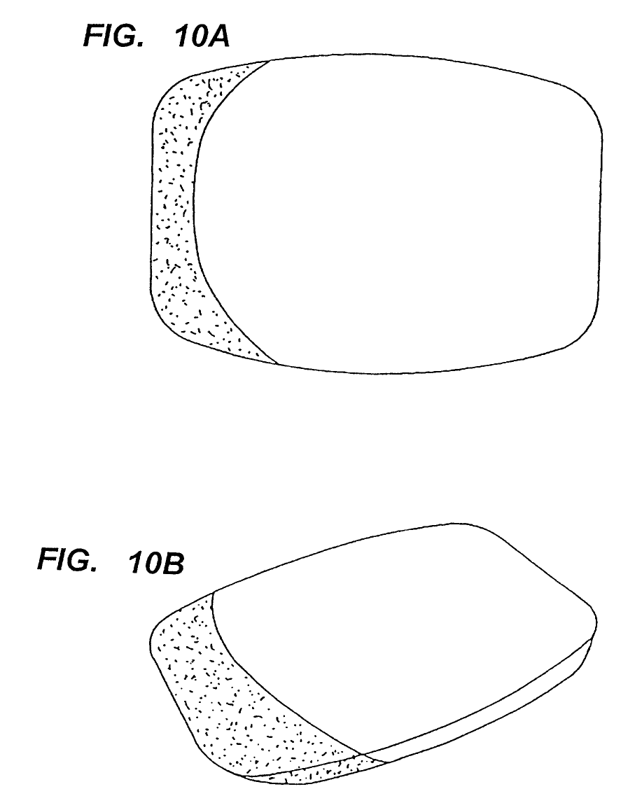

In a two-phase soap of the new diagonal type, the quantitative ratio of the two phases is identical (irrespective of the cutting direction and the cutting angle) provided the cutting point goes through the middle point of the soap (FIGS. 5A and 5B transverse type central section and FIGS. 6A and 6B). If the cutting point in the case of a two-phase soap of the novel diagonal type lies outside the middle point, for example as a result of horizontal or vertical shifting of the sectional area, a bar of soap with differently sized phases results (FIGS. 10A and 10B, transverse type of displaced section).

Application test:

1. Durability test of different multiphase soaps

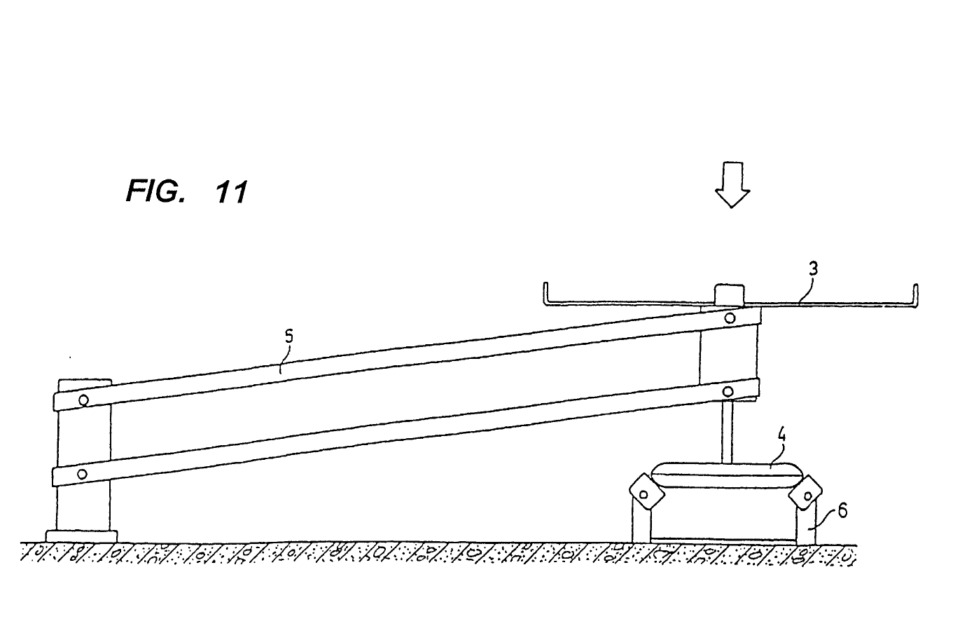

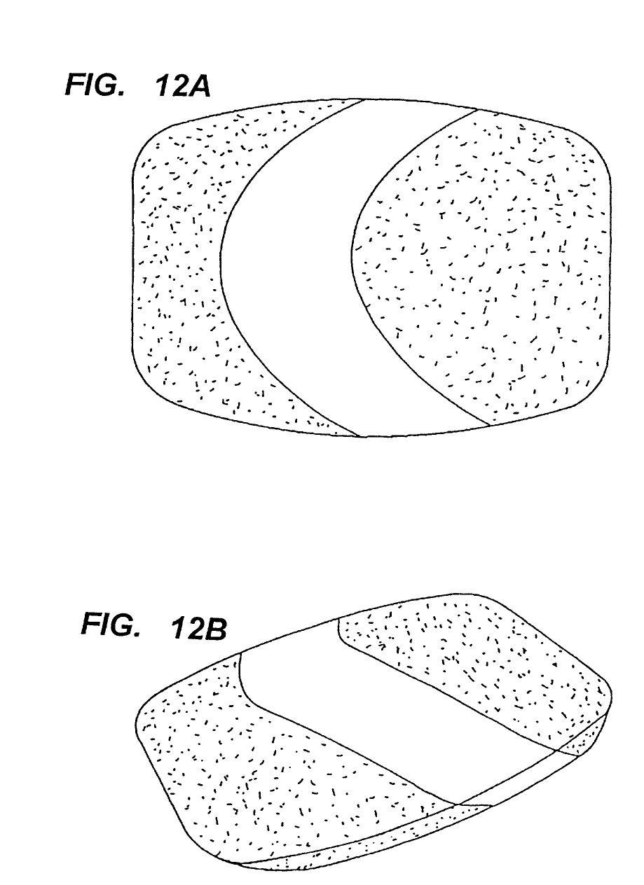

The mechanical durability of a soap is of importance for suitability during daily use. It is demonstrated that multiphase soaps with a diagonal section are more durable than would be expected on the basis of the sectional area and, in particular, more durable than soaps with a straight vertical cut (FIGS. 7A-7B, 8A-8B and 9A-9B). Using a device (FIG. 11), fracture experiments with weights were carried out on soaps having different types of design and different combinations of soap formulation. The device has a lever arm (5), which on one side has a plate (3) on which the weight may be placed to place strain on the soap (4). On the other side, the lever arm is pivotably mounted. The soap bar (4) is supported by a flexible holding device (6). The device loads the bar of soap (4) in the center in order to simulate stress in daily use, e.g., simple falling to the ground. The bars of soap were loaded in chronological order of ten seconds in each case with weights from five kg upwards in 0.5 kg steps. Provided a weight was held, the soap was loaded with a further weight until fracture of the soap occurred.

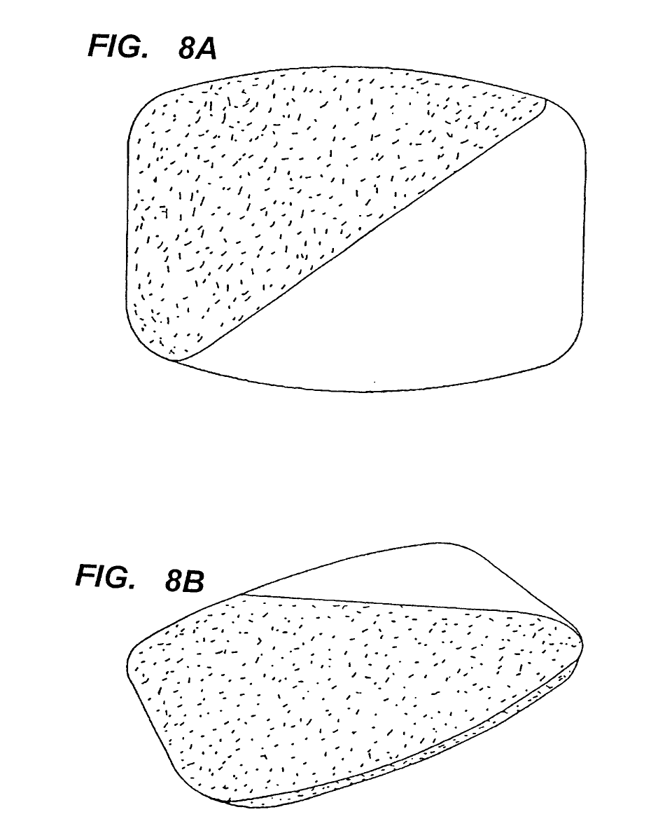

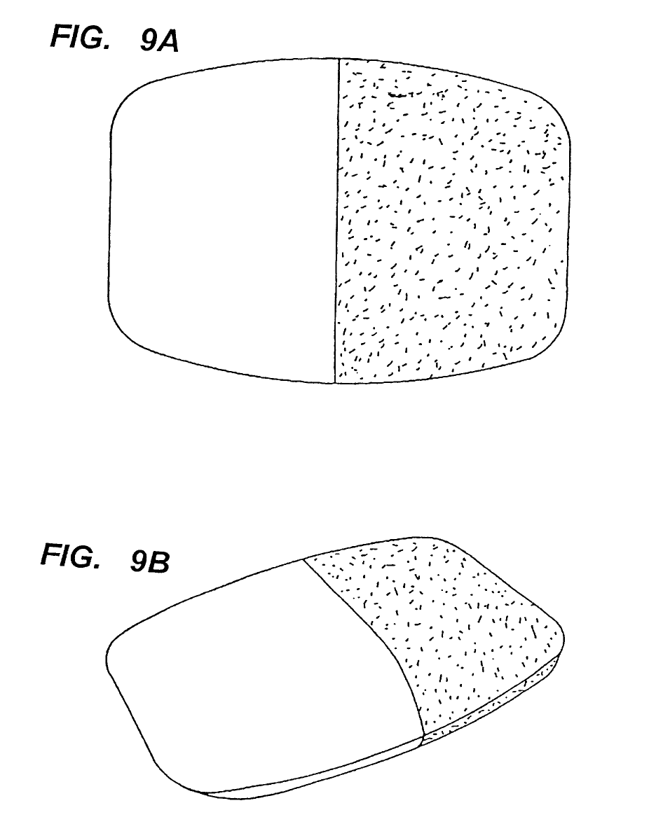

TABLE 1
Fracture stability

| | Fracture | Standard | Relative standard |
|---|---|---|---|
| | weight | deviation | deviation |
| Type | [kg] | [kg] | [%] |
| T1 FIGS. 1A/1B | 20 | 1 | 5 |
| T2 FIGS. 9A 9B | 7.5 | 0.5 | 7 |
| T3 FIGS. 7A 7B | 9.5 | 0.5 | 5 |
| T4 FIGS. 2A 2B | 19.5 | 0.75 | 4 |
| T6 FIG. 8A 8B | 8.5 | 0.5 | 6 |
| T7 FIGS. 3A 3B/58° | 12.5 | 0.5 | 4 |
| T8 FIGS. 3A 3B/38° | 15.5 | 0.75 | 5 |
| T9 FIGS. 3A 3B/35° | 20.5 | 0.75 | 4 |

It can be seen that the types of soap with the diagonal design are significantly more loadable, and there is therefore considerably greater durability in daily use. The results of the fracture tests were confirmed by consumers during daily use.

2. Preference test with regard to design

Consumer preference was carried out in a comparison test with the three soap types of horizontal section (FIGS. 2A and 2B), vertical section (FIGS. 9A and 9B) and the diagonal section according to the present invention (FIGS. 3A and 3B). Of the 100 people questioned, three of those questioned preferred the vertical sectional type, four of those questioned preferred the horizontal sectional type and 93 of those questioned preferred the novel diagonal sectional type. This means that the novel design was chosen with a significance of >99.9%.

Because of the novelty of the harmonious combination of two soap phases, there is interest in an application.

3. Preference test with regard to scent

Consumer preference for scent was carried out in a comparison test with two two-phase soap bars: a two-phase soap bar A (equal proportions by area, diagonal section through the central point, FIGS. 3A and 3B) with two different perfumings was compared against another soap bar B of identical construction which had been perfumed with a 1:1 mixture of these two compositions. The perfume concentration was 1% in both bars of soap.

It was found that the scent is more intensive in the soap bar with the separate perfume oils A. Both perfume oils were perceived in parallel. The scent gains an additional dimension.

4. Formulation

In addition to the perfume oil, soaps also comprise active ingredients, such as cooling substances, UV filters, antibacterial active ingredients, deodorizing active ingredients and others. These active ingredients are frequently expensive and are therefore only incorporated into soaps in small amounts. The concentration of the individual active ingredients is often below the limit of effectiveness. In the novel two- or multiphase soap it is possible to incorporate such an active ingredient in a targeted manner into one of the soap phases. As a result of the concentration of active ingredients in one part of the soap, increased effectiveness is achieved in the case of targeted application of one soap phase.

5. Recognition tests

To achieve a visible effect in a case of a multiphase soap, it must be possible to distinguish a certain proportion of the two phases when the soap viewed from above or from a customary viewing angle of about 45°. Three tests for the spontaneous recognition of multiphase soaps are described below.

The first soap phase is defined as the soap phase with the largest visible portion based on the area of soap projected in top view. The second soap phase is the soap phase with the second largest visible proportion. The visible proportion of the second and subsequent soap phases is expressed as a ratio of the projected area of the soap relative to the first soap phase or as a percentage of the overall area.

Tests for soaps having the new diagonal type: to determine the spontaneous recognition effect of multiphase soaps, six different test groups each of 20 participants were shown the bar of soap to be assessed (color combination green/white, FIGS. 3A and 3B) for three seconds viewed from above from a distance of one meter. The participants were then asked about what they had seen and the number of different soap phases.

In these consumer-oriented tests, it was found that a very good recognition effect exists for the diagonal soap type (transverse type second phase at one end) if, in top view, the area ratio of first to second soap phase is about 5.6:1 or if the proportion of the second soap phase of the overall area is about 15%. Below a proportion of 10%, the spontaneous recognition decreases significantly (see Table “recognition rate of multiphase soaps experiment 2”). A poor recognition effect is found if a two-phase bar of soap with the horizontal design (FIGS. 2A and 2B, color combination green-white) is viewed from a viewing angle of about 45°. In the case of this type of two-phase soap, no recognition of the two phases is possible when viewed directly from above. At a viewing angle of 45° a ratio of 1:12.5 or about 8% of the visible area for the second soap phase likewise arises.

Table 2

Recognition of two or more phases of a soap (FIGS. 3A and 3B diagonal type according to the present invention) as a function of the area ratios of the individual soap phase in top view.

| Second to | Second phase | | |
|---|---|---|---|
| first phase | area percent of | | Phases not |
| area ratio | overall area | Phases recognized | recognized |
| 1:20 | 5 | 4 | 16 |
| 1:10 | 10 | 8 | 12 |
| 1:6.7 | 15 | 16 | 4 |
| 1:5 | 20 | 18 | 2 |
| 1:4 | 25 | 20 | 0 |
| 1:3.3 | 30 | 20 | 0 |

Formulation section for different types of soap

For the preparation of multiphase soaps it is possible to use, as soap bases, e.g., alkali metal soaps, syndets or combinations of the two. In the case of all combinations of the soap bases, the water content of the individual soap formulations is to be taken into consideration. Because of the varying shrinkage of the individual soap formulations, separation at the contact surface and thus breaking of the soap may arise. By suitably adjusting the water content in the individual soap formulations and by virtue of the new diagonal design it is possible to use numerous combinations of soap combinations for the preparation of stable multiphase soaps.

Since the so-called solid skin-cleansing composition can, by virtue of different additives and a special preparation process, also be prepared in transparent or opaque form, it is possible to prepare a very wide variety of combinations, including, of course, colored ones.

Using the multiphase soaps, scent accords can be represented which would lead to discolorations in purely white soaps. During the preparation of multiphase soaps, the perfume oil constituents which may lead to discolorations are taken up in the colored part. Perfume oil constituents which tend toward clouding in transparent soap are taken up in the opaque or nontransparent phase.

Preparation process:

The preparation of soaps is known (Soaps and Detergents, Luis Spitz, 0-935315-72-1 and Production of Soap, D. Osteroth, 3-921956-55-2). The preparation of the novel multiphase soaps was carried out as described in the process below as example: first, the soap bases are admixed with the above-described additives, such as perfume oil, cosmetic ingredients, dyes, stabilizers and further additives, and then milled. The soap composition was then extruded at a jacket temperature of about 22° C. and a head temperature of about 45° C.

The resulting soap strands are then cut to the soap shape. The same is carried out for the soap strands of the second soap phase. The two soap strands are then cut in parallel and diagonally corresponding to the subsequent cutting shape and design type at an angle of from 14° to 70°. Prior to the stamping operation, the soap strands prepared in this way are aligned by means of the soap mold. The stamping operation was carried out, depending on the type of soap stamping machine used, with a pressing weight of from about 1.0 to 2.0 t or a pressing force of from 4 to 10 bar. During this stamping operation, both soap compositions have a temperature of from about 40 to 50° C.

Reference number list

In FIGS. 2-4,

Ref No.

1. Phase 1.

2. Phase 2.

In FIG. 11,

3. Lever arm

4. Plate

5. Weights for loading onto the soap

6. Rotatable mounting of the lever arm

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A soap comprising two or more phases, wherein said two or more phases are visible when viewed from above and from the side.

2. A soap according to claim 1, wherein each phase is visible in the vertical, longitudinal and transverse projection to at least 15% based on the overall projected area.

3. A soap according to claim 2, wherein each phase is visible in the vertical, longitudinal and transverse projection to at least 20% based on the overall projected area.

4. A soap according to claim 1, wherein the areas of the adjacent phases are cut diagonally and cambered towards one another.

5. A soap according to claim 1, wherein each phase comprises different perfume oils, cosmetic ingredients, dyes and/or further additives.

6. A process for the preparation of multiphase soap comprising two or more phases, wherein said two or more phases are visible when viewed from above and from the side, comprising the step of diagonally cutting in the form of soap strands the individual phases at an angle of from 14° to 70°, and the points of intersection are joined under pressure so that cambering arises at the points of intersection.

7. A process according to claim 6, wherein the angle is 30° to 55°, and the points of intersection are joined under pressure so that cambering arises at the points of intersection.
